(19) United States
(12) Patent Application Publication
(10) Pub. No.: US 2009/0210049 A1
(43) Pub. Date: Aug. 20, 2009

(54) Peripheral overlap stent

(76) Inventors: Joseph Michael Thielen, Buffalo, MN (US); William Joseph Drasler, Minnetonka, MN (US)

Correspondence Address: Joseph M. Thielen, 3027 Cameron Ave. S.E., Buffalo, MN, 55313, US

(21) Appl. No.: 12/378,081

(22) Filed: Feb. 11, 2009

Related U.S. Application Data
(60) Provisional application No. 61/065,913, filed on Feb. 15, 2008.
(60) Provisional application No. 61/066,039, filed on Feb. 15, 2008.

(51) Int. Cl. A61F 2/06

(52) U.S. Cl. 623/1.16; 623/1.2

(57) ABSTRACT

A peripheral stent with individual segments reduces the occurrence of fatigue fracture failure seen in vessels and tubes having bending and twisting movement. Segments can be attached via connecting fibers that biodegrade and offer the segments freedom of movement. The segments are balloon-expandable but will not be crushed by external forces placed upon the stent. Hinges and struts provide the stent with a plastic deformation during expansion and remain elastic if exposed to an oval shape. The segments overlap each other to provide improved scaffolding of the vessel wall and a greater flexibility during delivery. A composite stent having both balloon-expandable and self-expanding character has application in the venous system.

### CROSS REFERENCE TO RELATED APPLICATIONS

This invention makes reference and thereby includes aspects of issued U.S. Pat. Nos. 6,421,763; 6,312,460; 6,475,237; 6,451,051 which describes stents and attachment means having hinges and struts. This patent application also makes reference to two provisional applications filed 15 Feb. 2008 by Joseph M. Thielen: Overlap Stent with application number 61065913 and Segmented Stent with application number 61066039.

### BACKGROUND OF THE INVENTION

1. Field of Invention

This invention pertains to stents delivered via an interventional catheter into tubular vessels of the body such as arteries or veins in a small diameter conformation and expanded to a larger diameter to hold the vessel outward and allow passage of fluid such as blood.

2. Description of Prior Art

Stents used in specific vessels of the body such as the superficial femoral artery (SFA), carotid artery (CA), iliac artery, popliteal artery, other arteries of the lower leg, iliac and femoral veins, and other vessels that can be exposed to external forces that can cause vessel or stent deformation are generally required to be self-expanding stents. Self-expanding stents will return to their generally round cross sectional shape if the vessel they are located within is exposed to a force or movement that causes the shape to momentarily become oval or flattened. Standard balloon-expandable stents will remain in a crushed shape if they are exposed to such an external force and hence are not used in SFA and CA stenting and stenting in other vessels of the peripheral vasculature and tubules of the body. Coronary stents however can be formed from a more plastically deformable metal since external forces from outside the body cannot transmit well to the coronary vessels of the heart. Thus standard balloon-expandable stents can be used in coronary applications.

A self-expanding stent is typically delivered via a delivery sheath that holds the stent in a small diameter conformation during delivery, and the stent is released from the delivery sheath once the stent is delivered to the site of the lesion. The accuracy of placing a self-expanding stent via an external delivery catheter is not as accurate as the physician often would like because the stent can often jump out of the delivery catheter as it expands out to its final deployed diameter. The delivery sheath itself can also add not only stiffness to the delivered system but also can add to the profile of the overall stent system.

The delivery sheath can also scrape or abrade any drug or coating that may be applied to the outside surface of the stent as the sheath is removed during the delivery of the stent to the lesion site. Self-expanding stents have also been known to migrate through the wall of an artery or vein due to the continued expansion force being applied by the stent. Also, self-expanding stents do not exert a significant holding force outward when the stent is in its fully expanded configuration. The outward holding force to resist an external crushing force is only adequate after the stent has been forced to reduce in diameter allowing its holding force to increase and balance the external force.

The stent wall structure must have enough axial compressive strength such that upon release of the stent from the sheath into the vessel the stent retains its axial length. This compressive strength is often supplied by connectors that can connect individual ring-like segments of a stent together to form the entire stent. Often fractures can occur at the junction of these connectors with other stent metal elements.

Balloon-expandable stents can be delivered with more accuracy within the lesion of a blood vessel. Balloon-expandable stents, if designed correctly, can allow a more flexible delivery system for a stent because the tubular members of the balloon catheter are all smaller than the stent and can be made more flexible than a delivery sheath found in a self-expanding delivery system. Balloon-expandable stent systems can also allow a smaller profile than the delivery sheath for a self-expanding system. This is because most balloon-expandable stents in a nondeployed state have a lumen diameter whose minimum size will generally accommodate easily the space required by the balloon portion of a balloon dilation catheter. Thus the balloon delivery catheter for a balloon-expandable stent does not actually add to the profile of the balloon-expandable stent delivery system, however, the delivery sheath of a self-expanding stent delivery system can add to the overall self-expanding stent system profile.

SFA stents, carotid stents, coronary stents, venous stents, other peripheral stents, and stents in general require a minimum amount of stent strut surface area percentage to provide an optimal result. If the surface area of the expanded stent is too low, the plaque of a diseased vessel will not be properly supported by the struts and one can anticipate larger than normal thrombosis due to plaque protrusion between the struts. If too much strut surface percentage is present, one can get excessive thrombosis due to the exposure of blood to excess foreign material. Therefore care must be given to ensure proper scaffolding of the vessel wall but not to an excess.

Stent strut fracture can lead to a tissue site associated with excessive tissue hyperplasia leading to possible restenosis as a result of the strut fracture. A broken strut or a connector located between segments of a stent can often cause an inflammatory response due to continued relative movement with respect to the tissue and result in hyperplastic tissue growth. Such strut fractures typically occur due to movement within the vessel such as bending, twisting, and stretching. Often the fracture occurs at the site of junction of a connector with one of the stent ring-like segments. A stent design should allow for such movement to occur within a vessel and stent without focusing the movement to a specific location within the stent structure leading to strut or connector fracture failure.

### SUMMARY OF THE INVENTION

The present invention overcomes many of the obstacles described above for SFA, Carotid, Coronary, or other stent designs. The stent of one embodiment of the present invention is comprised of a number of hinges and struts that form the wall structure of the stent. The struts form the elongated regions of the stent wall structure and the hinges form the junction region for these struts.

The hinges can be designed to allow the deformation associated with the expansion to be focused and thereby allow the stent to be formed out of an elastic metal such as nitinol and yet be balloon-expandable. The expansion deformation is focused by providing a hinge width and length that is relatively small, smaller than the strut width. The hinges can alternately be formed from metals that are more able to undergo plastic deformation such as stainless steel, platinum, and other alloys. The hinges have a larger radial dimension than other parts of the stent and which then protrude from the outer surface and give the outer surface of the stent a nonuniform shape. The larger hinge radial dimension provides a resistance to bending in the plane of the stent surface due to a crush deformation.

The struts are designed with a larger strut width that will not allow bending to occur in the plane of the stent surface. Additionally the struts are formed with a thin radial dimension that allow the strut to be bent into an oval shape during a crush deformation and return back to a round cross sectional shape. The strut radial dimension is thinner than the hinge radial dimension. The strut can be formed out of an elastic metal such as nitinol or Elgiloy or it can be formed out of a more plastically deformable material such as stainless steel and rely on a thin radial dimension to remain elastic. A transition region serves to form a gradual transition from the hinge dimensions to the strut dimensions. Thus the stent of this embodiment with properly designed hinges and struts can be balloon-expandable and non-crushable. The stent when formed out of stainless steel or other metal alloys can be designed to focus plastic deformation into the hinge and be designed with a thin strut that will remain elastic during a crush deformation. The stent can further be formed out of a biodegradable material if desired because the stent structural properties are determined by the dimensions for the hinges and the struts. Such biodegradable materials include but are not limited to polylactic acid, polyglycolic acid, polyethylene glycol, collagen, magnesium, and other polymeric based or tissue based materials.

In one embodiment the stent is formed of a series of tapered rings that overlap one another along its axial length during delivery. Each ring can have a modified form of zig-zag geometry but with the struts generally nonparallel to each other due to the taper and also having specially designed hinges and struts to provide the balloon-expandable and non-crushable characteristics. These overlapping stent segments give the inner and outer surfaces of the stent a stepped shape that is not locally cylindrical. Overlapping the segments in the axial direction provides the stent with a greater amount of strut material that can be available to provide scaffolding to the vessel wall after stent deployment than can be accomplished without the overlapping. Also the stepped inner surface provides for improved securement of the stent onto the balloon portion of a balloon catheter during the delivery of the stent to the lesion site. In the deployed state each segment is closely nested next to its neighboring segment to provide improved scaffolding of the vessel wall. This will allow the present stent to be less prone to thrombosis and reduce the amount of emboli generated due to poor scaffolding or from thrombosis. The overlapping also improves flexibility by preventing the intersection of the ends of neighboring segments during delivery.

In another embodiment alternating segments are positioned with both segment ends either below or above a portion of its neighboring segments such that the segments remain parallel to each other in the non-deployed state. Thus every other segment is delivered with a smaller diameter than its neighboring segments on each end which have a larger diameter. The outer surface therefore has a generally stepped appearance from a smaller diameter to a larger diameter and back to a smaller diameter etcetera as one moves axially from one segment to another. The stepped inner surface improves securement to the balloon portion of a balloon delivery catheter in its non-deployed state.

Additionally, the overlapping allows each stent segment to move more easily relative to its neighboring segment without intersection during a bending deformation and thereby providing the stent with greater flexibility during delivery. In the deployed state the individual segments allow the stent to be very flexible with respect to a bending or twisting deformation. The segmented structure for the stent which allows the individual movement of one segment to move with respect to another in the deployed state further reduces the tendency for fatigue fracture to the structural elements of the stent.

In one embodiment the individual segments are connected to each other via joining elements that are spacing members that provide the stent with the stability and integrity during deployment and in the deployed state to keep the segments aligned and evenly spaced. The spacing members can be straight or curved to allow for more bending deformation. They can be formed from metals already described and can be contiguous with the elongated elements or junctional regions of the wall structure.

In another embodiment the individual segments are connected to each other via joining elements that are thin connecting fibers that are woven, twisted, tied, or adhered to a segment and attach the segment to its neighboring segment. The connecting fibers can be biodegradable fibers that will either degrade or dissolve in the body in a period of days or weeks. The fibers can be multifilament fibers such that they are very flexible and do not provide a compressive strength. Unlike the connectors for current self-expanding stents which have more substantial connectors that supply a compressive strength to align the stent coming out of the sheath, the connecting fibers of the present invention do not require this compression resistance characteristic since the stent is being delivered by a balloon catheter. In one preferred embodiment the connecting fibers are biodegradable. In another embodiment the connecting fibers are formed from flexible multifilament polymeric or metallic materials that are also very flexible but are not biodegradable.

One embodiment for the stent is a balloon-expandable stent having a profile that is lower than that of a self-expanding stent. In one embodiment an SFA or carotid stent can be delivered through a smaller guide catheter from a femoral artery or radial artery approach. The balloon-expandable stent of this embodiment can be delivered with more precision than a self-expanding stent.

In another embodiment, the balloon-expandable stent can be delivered to the SFA artery, Coronary artery, or other vessel via a balloon catheter with a drug such as Taxol or Sirilomus deposited directly or loaded into a carrier polymer that is coated onto at least a portion of the stent surface or delivered via other deposition methods. An external sheath is not needed as is required for most self-expanding stent systems used in the SFA or other peripheral vessels of the leg or iliac artery or vein; such an external sheath can make delivery of drug eluting stents more difficult due to abrasion of the drug or stent coating.

Machining for the stent of the embodiment having hinges and struts that provide balloon expandability and noncrushability generally requires that a contoured external shape that is not purely a cylindrical surface be machined into the external surface of a tube. If the tube is a metal tube such as nitinol or stainless steel, the external contour can be machined via a variety of methods including standard machining, EDM, laser, waterjet, or laser plus waterjet. The same types of machining methods can be used to remove material in a radial direction to form junctional regions such as the hinges, transition regions, and the elongated elements such as the struts.

In yet another embodiment a balloon-expandable stent can be formed with a wall structure that comprises standard elongated elements and junctional regions instead of the specifically designed hinges and struts described above. The stent could be used in applications such as the coronary artery or other vessels that work well with balloon-expandable stents and are not exposed to external crush forces. The geometry for the stent is comprised of a series of segments that are joined via joining elements that are either connecting fibers or spacing members. Each segment can have a geometry that is similar to existing wall structures such as zig-zag, closed cell, or other combinations of cell structure. One embodiment does include the presence of overlap regions in the axial direction between neighboring segments. Connecting fibers are woven, tied, or attached to join each segment with a neighboring segment as described earlier. Alternately, spacing members that are contiguous with the stent segments can connect individual segments together. In another embodiment each segment can be tapered as described for the hinge stent design where each segment extends under one of its neighboring segments and over another of its neighboring segments forming overlap regions. Alternately each segment can be either of a larger diameter or a smaller diameter arranged such that every other segment is either of large diameter or small diameter. The larger diameter segment thus overlaps over the smaller diameter segments. Alternately individual segments that do not overlap can be joined via connecting fibers that are flexible. This embodiment can be formed from materials such that the stent is balloon-expandable.

Metals more capable of undergoing plastic deformation such as stainless steel, titanium, platinum, and other alloys can be used to form a balloon-expandable stent. The overlap region present when the stent is in a nondeployed state provides the advantage of improved scaffolding when the stent is in its deployed and larger diameter state. The overlapping also allows the stent to be more flexible in its nondeployed state and helps to secure a balloon-expandable stent to its underlying balloon during deployment.

In still another embodiment a self-expanding stent can be formed with any of the wall structures described for the balloon-expandable embodiments. A self-expanding stent may not offer some advantages provided by the balloon-expandable stent described herein, however for those applications where profile and placement accuracy can be accommodated, a self-expanding stent may be of significant value. Specific peripheral vessels of the leg or neck for example could benefit from such self-expanding stents. For the embodiment that includes the specifically designed hinges and struts the hinge can be dimensioned such that it remains elastic during expansion deformation. The hinge portion of the self-expanding stent would require an increased hinge length to unfocus the deformation it is exposed to during stent expansion. Materials for a self-expanding stent include the elastic metals such as nitinol and elgiloy, and other materials such as stainless steel, biodegradable metals and polymers.

Another embodiment of the present invention is well suited to applications where one portion of the stent is formed as a balloon-expandable portion and another portion is self-expanding. Such a composite stent can have application in a variety of tubular vessels of the body including veins, esophagus, trachea, intestine, bile ducts, urinary tracts, as well as arteries and hollow organs and tubules of the body. A variety of applications that would benefit from a stent of this design are in the venous system of the body. One example is the left common iliac compression syndrome. Here the iliac artery places a compression force upon the iliac vein causing it to become compressed and leading to thrombosis or stenosis in the vein.

Standard self-expanding stents do not work well in many venous applications because self-expanding stents at their native expanded diameter do not exert a large outward force making them prone to compression from external forces including compression forces from an adjacent or nearby artery. If the self-expanding stent is made at a larger diameter but deployed into a vein or other vessel of smaller diameter, then it risks migration of the stent struts through the wall of the blood vessel or other tubular member of the body. A balloon-expandable stent is designed to exert zero outward force at its expanded configuration but is unable to extend out further if the vein diameter should enlarge and hence can result in embolization of the stent. Placing a self-expanding portion at either end of a balloon-expandable stent can overcome the problem associated with stent embolization. The self-expanding portion can be formed such that it has a very large native diameter but that the outward force is very low. Thus the self-expanding portion of the stent will not have a desire to migrate through the wall of the vessel but will act to hold the stent against the vessel wall to prevent embolization of the stent.

The present composite stent embodiment can be constructed out of a single material including but not limited to nitinol, Elgiloy, or stainless steel such that the balloon-expandable portion located in the central portion of the stent is non-crushable. The balloon-expandable portion and the self-expanding portions can be made contiguously if desired since the balloon-expandable stent properties are obtained by the dimensions of the hinges and struts of the balloon-expandable portion. The hinge and strut structure of the present invention provide the balloon-expandable portion with the ability to be made out of a generally elastic material but still undergo a plastic deformation of the hinges. The struts are formed with a thin radial dimension to remain elastic during a crush deformation. The self-expanding portions located at each end region of the stent are formed from a standard self-expanding design such as the zig-zag design or from a hinge design that allows the hinge to provide a self-expanding character to the stent. The individual segments can be overlapped or can lie adjacent to each other and can be of an open or closed wall structure. The self-expanding portions can have joining elements that are either spacing members to join individual segments together or connecting fibers can be employed.

Additionally, the stent segments of the self-expanding and the balloon expanding regions can be formed of different materials, such as an elastic material for the self-expanding region and a ductile material for the balloon expanding region. The segments can be connected together with joining elements that are either connecting fibers or spacing elements. The connecting fibers can be biodegradable filaments that degrade over a period of time that can be determined by fiber composition and physical size.

### BRIEF DESCRIPTION OF THE DRAWINGS

FIG. 1A is an isometric view of the stent having tapered segments and connecting fibers.

FIG. 1B is an isometric view of the stent having tapered segments and connecting fibers attached to the struts.

FIG. 2 is an isometric view of a hinge, a strut, and a transition region.

FIG. 3A is a plan view of two tapered segments of the stent.

FIG. 3B is a plan view of two tapered segments of the stent attached by a curved spacing element.

FIGS. 4A-4B are plan views of junctional regions of a stent being attached to a filament.

FIG. 4C is a plan view of a stent strut being attached to a filament.

FIG. 5A is an isometric view of a stent having hinges and struts and connecting fibers in an expanded configuration.

FIG. 5B is an isometric view of a stent having hinges and struts in an expanded configuration after the connecting fiber has degraded.

FIG. 6 is a plan view of a stent having hinges and struts and spacing members that has been cut open and flattened in an expanded configuration.

FIG. 7A and 7B are plan views of a portion of a stent having hinges and struts in a partially deployed configuration.

FIG. 8 is an isometric view of stent having hinges and struts and tapered segments held together by connecting fibers and having a drug or coating on the struts.

FIG. 9 is an isometric view of two tapered segments of the stent being held together by spacing members.

FIG. 10A is an isometric view of a stent with hinges and struts and having outer and inner segments being held together by connecting fibers.

FIG. 10B is an isometric view of a stent with hinges and struts and having individual segments with axial space between them and held together by connecting fibers.

FIG. 11 is a plan view of a stent having hinges and struts and having inner and outer segments held together by spacing members.

FIG. 12 is a sectional view of the overlap region of two segments of the stent shown in FIG. 11.

FIG. 13 is an isometric view of a stent having a standard zig-zag structure but having tapered segments with overlap regions joined together by connecting fibers.

FIG. 14A is an isometric view of a stent having standard zig-zag structure but having tapered segments with overlap regions joined together by spacing members.

FIG. 14B is an end view of a tapered segment shown in FIG. 14A.

FIG. 15 is a plan view of two tapered segments having a closed cell configuration and overlapped with each other and held together by spacing members.

FIG. 16 is a plan view of an inner'segment and two outer segments that have closed cell configuration and are overlapped with each other and held together by spacing members.

FIG. 17 is an isometric view of a stent having a standard zig-zag open cell construction but having overlap of inner and outer segments and being held together by connecting fibers.

FIG. 18 is a side view of a stent having a standard zig-zag open cell construction but having overlap of inner and outer segments and being held together by spacing members.

FIGS. 19A and 19B are plan views of the hinge and strut portions of a stent that is self-expanding.

FIG. 20 is an isometric view of a composite stent having a central portion that is balloon-expandable and two end portions that are self-expanding.

FIG. 21 is a partially sectioned side view of a composite stent loaded upon a balloon dilatation catheter and contained within an external sheath.

FIG. 22 is a side view of the composite stent of FIG. 20 in a deployed configuration.

### DETAILED DESCRIPTION OF THE INVENTION

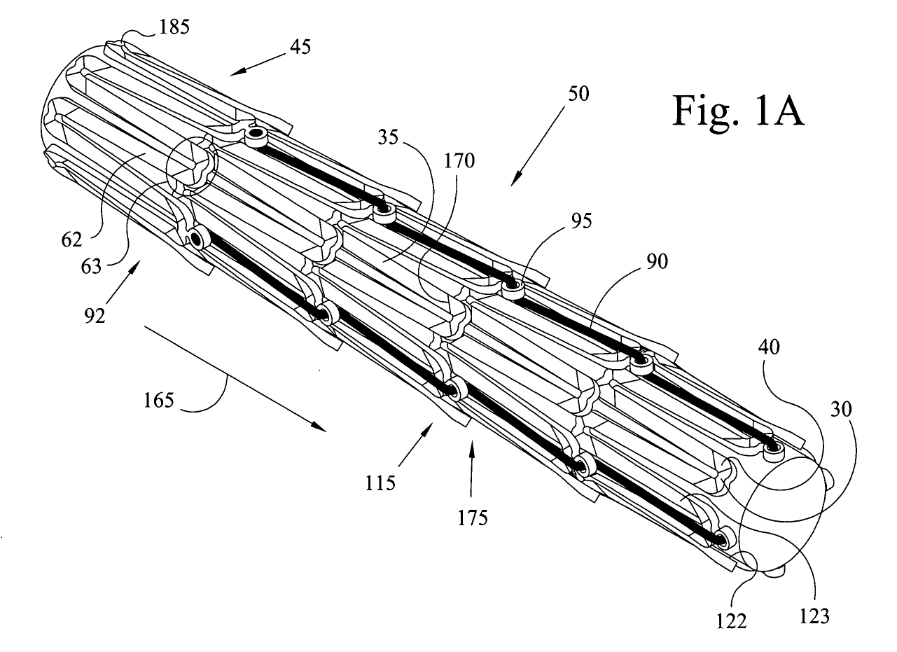

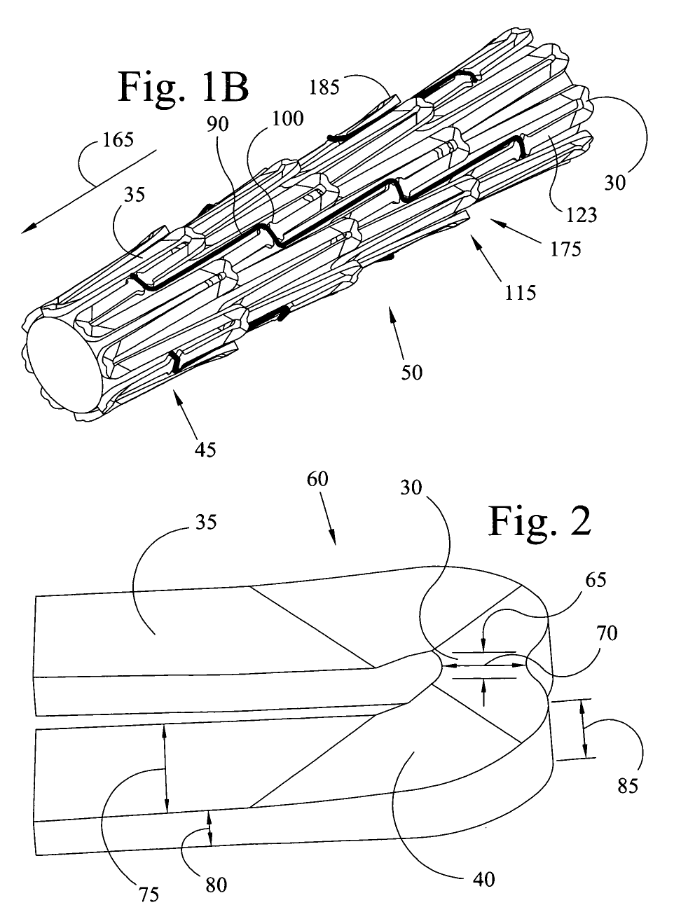

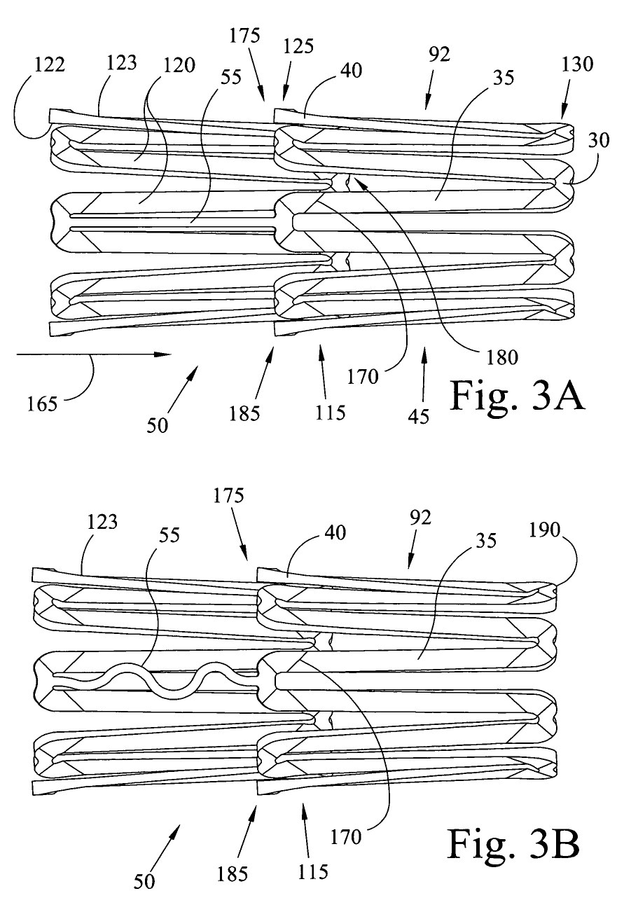

Embodiments of the present invention have a plurality of hinges (30) and struts (35) that are connected together via transition regions (40) as shown in FIGS. 1A, 1B, and 2. The embodiments of FIGS. 1A and 1B have joining elements (42) that are connecting fibers (90) to connect segments (45) of the stent (50). Alternate embodiments have joining elements (42) that are spacing members (55) to connect segments (45) as shown in FIGS. 3A and 3B. The struts (35) form the elongated elements (62) of the stent wall structure (60) and the hinges (30) and transition regions (40) form the junctional regions (63) of the wall structure (60) where one elongated element (62) joins with another elongated element (62). During deployment the hinge undergoes plastic deformation due to a small hinge length (65) and hinge width (70) (see FIG. 2) that focuses the expansion deformation as the stent (50) is exposed to a balloon dilation or other mechanical dilation. The strut (35) has a very large strut width (75) that resists deformation in the plane of the stent surface during the expansion of the stent (50). The strut (35) has a very small strut radial dimension (80) which allows the strut (35) to bend elastically if the stent (50) is exposed to an external crush force that causes the stent to form an oval shape. The hinge has a very large hinge radial dimension (85) that does not allow it to deform if it is exposed to an external crush force. The stent of the present invention is therefore able to be balloon-expandable but non-crushable.

The stent can be made of an elastic metal such as nitinol, Inconel, elgiloy, or other elastic metals or alloys. The focusing of the expansion deformation by the hinge will require that even elastic metals will undergo a plastic deformation in that region. Alternately, the stent can be constructed from other standard metals used in standard balloon-expandable stents such as stainless steel, platinum alloys, and other metals and alloys. The thin strut radial dimension will provide the ability for such metals to remain elastic during a crush deformation. Alternately, the stent can be constructed out of biodegradable materials such as poly L-lactic acid, polyglycolic acid, polyglycolic lactic acid, Polyurethane, polyethylene glycol, polycarbonate copolymers and a variety of other biodegradable materials. Biodegradable metals such as magnesium and other alloys can also be used to construct the present stent. The dimensions for the hinge and strut can be tailored to provide the focused deformation of the hinge and the elastomeric character of the strut during a crush deformation. The expansion forces, vessel holding forces, and crush forces can also be tailored to provide the desired characteristics.

A close-up of the hinge (30), strut (35), and transition region (40) is shown in FIG. 2 for the balloon-expandable embodiment. For a stent constructed out of nitinol or stainless steel the hinge can have a hinge radial dimension (85) of approximately 0.004 inch and ranging from 0.003 to 0.007 inch. This hinge radial dimension (85) resists bending in a radial direction. The strut (35) can have a strut radial dimension (80) of approximately 0.0015 inch and ranging from 0.0010 to 0.004 inch (for a stent of diameter ranging from 3-10 mm) and is designed to allow the strut (35) to always remain elastic when exposed to a crush deformation. The hinge and strut dimensions can vary beyond this dimensional range depending upon the diameter of the stent. The strut radial dimension (80) is generally lower for a plastically deformable metal in order to prevent plastic deformation of the strut (35) if exposed to a crush deformation that would make the stent become temporarily oval or flat. The hinge radial dimension (85) is larger than the strut radial dimension (80) and can vary depending upon the material of choice and the forces that are desired for expansion crush resistance, vessel outward holding force, and other stent requirements. The hinge width (70) is less than the strut width (75). For a balloon-expandable stent the hinge length (65) would be less than the strut width (75) to focus the deformation of the hinge (30) during stent expansion and provide a plastic deformation of the material of the hinge (30). The hinge length (65) should preferably be less than twice the hinge width (70) to provide a greater focusing of the deformation during expansion and could be similar or smaller than the hinge width (70) to provide even more focused deformation.

For the balloon-expandable embodiment the strut width (75) can have a dimension of approximately 0.006 inch and can range from 0.0035 to 0.010 inch. This large strut width (75) resists bending in the plane of the surface of the stent during deployment or expansion of the stent. The hinge width (70) controls the expansion force and is less than the strut width (75). The hinge length (65) for a balloon-expandable stent must be short in order to focus the expansion deformation with a length of approximately 0.003 inches (range 0.002-0.020 inch). This focusing of the deformation provides the stent with a plastic deformation and allows the stent to be balloon-expandable regardless of the material of which it is constructed. A transition region (40) is provided to form a gradual transition of dimension from the hinge (30) to the strut (35). The dimension for the hinges (30) and struts (35) can be generally increased to provide the necessary force requirements of the stent if it is constructed from a biodegradable polymer. A larger stent diameter can also require the hinge and strut dimensions to be adjusted to obtain the required forces.

In another embodiment the stent of the present invention could also be a self-expanding stent. To make a self-expanding stent with specifically designed hinges (30) and struts (35) the hinge length (65) must be made longer than for a balloon-expandable stent such that it does not focus the deformation that occurs during the expansion of the stent as it is released from the delivery sheath and is deployed. To allow the hinge (30) to remain elastic by not focusing its deformation during the expansion of the stent the hinge length (65) should be made longer than twice the hinge width (70). The hinge length (65) would preferably provide elastic behavior if the hinge length (65) were longer than the strut width (75).

The hinge length (65) for a self-expanding stent can be approximately 0.020 inch (range 0.008-0.060 inch). The material for a self-expanding stent can be Nitinol, Elgiloy, or other alloys. Stainless steel can be used provided that relatively longer hinge lengths are used. The outward holding force onto the vessel and the stent crush force can be tailored by adjusting the dimensions of the hinges (30) and struts (35) as described in the earlier referenced patents.

As shown in FIGS. 1A, 1B, 3A, and 3B the stent of these embodiments are formed of tapered segments (92) that are overlapping each other thereby making the shape of each tapered segment (92) into a gradual conical surface during delivery. It is understood that many such segments extend in an axial direction (165).

The embodiments shown in FIGS. 1A and 1B show tapered segments (92) that are joined together with connecting fibers (90). A connecting fiber (90) can be a biodegradable multifilament fiber that is very flexible and will dissolve or degrade in the body in a period of preferably a few days (range 3 days to several months). A biodegradable connecting fiber (90) can be made from polyethylene glycol, polylactic acid, polyglycolic acid, polycarbonate degradable copolymers, or other biodegradables used in the medical device industry for sutures, vascular closure devices, and other biodegradable implants. Other biodegradables that could be used include biodegradable metals including magnesium. Alternately the connecting fiber (90) can be made from a flexible polymer that is not rapidly degradable such as Dacron, polyethylene, polyurethane, or other polymer that can be formed into a flexible small diameter monofilament fiber or multifilament fiber. Additionally, a very thin metallic nonbiodegradable multifilament fiber could also be used.

FIG. 1A shows one embodiment of the present invention having the joining element (42) that is a connecting fiber (90) passing through an open element (95) attached to the hinge (30). The connecting fiber (90) extends axially connecting one segment with its neighboring segment and continuing on to join to the next segment. As shown there are four connecting fibers (90) however one could have between 2-8 connecting fibers (90) and they do not have to run axially as shown; rather other patterns can exist for the path of the connecting fibers (90). It is anticipated that a small amount of adhesive or biodegradable material placed at each site where the connecting fiber (90) passes through the open element (95) or makes contact with the stent segment could provide a secure attachment for the fiber to each segment. Alternately a knot or tie can be used to provide securement. Other methods of interfacing or attaching the connecting fibers (90) to the tapered segments (92) are also anticipated which do not require the use of an open element (95).

FIG. 1B shows another embodiment wherein the tapered segments (92) are connected by joining elements (42) that are connecting fibers (90) attached to the struts (35). In this embodiment one or more strut tabs (100) are formed onto the struts (35) to aid holding and attaching the connecting fibers (90). As will be shown later, one can secure a connecting fiber (90) to a strut (35) by twisting the individual filaments of a multifilament fiber around the strut (35) at the site of the strut tab (100). The connecting fibers (90) as shown in this embodiment form a gradual helical pathway as it extends along the outside of the stent (50). The connecting fibers (90) could also attach to the struts (35) via an adhesive or other bonding method.

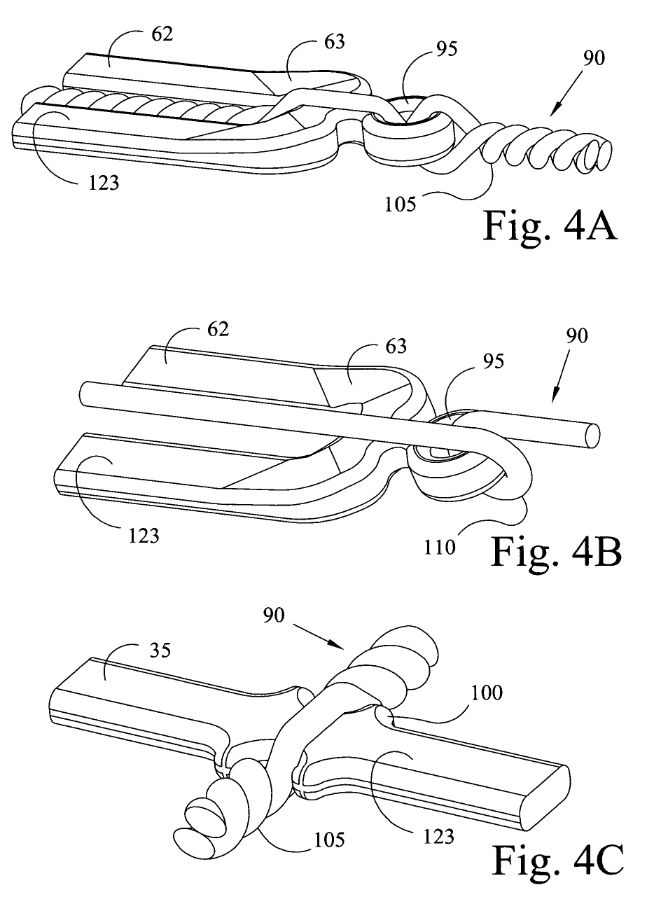

Several ways can be implemented to attach the connecting fiber (90) to the open element (95) as shown in FIGS. 4A and 4B. The connecting fiber (90) can be made of multifilaments wherein a portion of the filaments (105) pass through the open element (95) in one direction and the rest pass through in the other direction. As shown in FIG. 4A, the fiber is comprised of two filaments (105) that pass in opposite directions through the open element (95). The fiber filaments (105) are then twisted on each side of the open element (95) in the opposite direction to hold or secure the connecting fiber (90) to the open element (95). It is also possible to wind or tie the connecting fiber (90) around the open element (95) forming a loop (110) to provide securement of the connecting fiber (90) to the open element (95) as shown in FIG. 4B. The stent of the present invention is not required to have the hinge and strut structure shown in FIG. 2. The strut (35) can be represented as an elongated element (62) that is joined to another elongated element (62) at a junctional region (63) as shown in FIGS. 4A and 4B. The elongated element (62) can be contiguous with the junctional region (63). This structure is similar to that found in typical zig-zag design self-expanding stents currently found in the clinic.

The securement of the connecting fiber (90) to the strut (35) can be formed as shown in FIG. 4C. A portion of the filaments (105) of a multifilament connecting fiber (90) are passed around one side of the strut (35) between two tabs and the remaining filaments (105) are passed around the other side of the strut (35). The filaments (105) are twisted on each side of the strut (35) to form a fiber that can then continue on to the next strut (35) for securement.

Other methods of attaching the segments (45) together have been anticipated. Fibers can be formed of a polymeric or biodegradable material and applied in an adhesive manner to the outer surface (123) (see FIG. 3A) of the stent to hold the individual segments (45) into alignment after delivery to the vessel. For example, electrostatic spraying can be used to apply polymeric fibers such as silicone, polyurethane, collagen, polyethylene glycol, polylactic acid, polyglycolic acid or other fiber forming materials to the outside surface forming a web of fibers that would serve to hold the segments (45) in relative position. Other methods for applying fibers to the outer surface (123) of the segments (45) are also possible including extrusion or bonding of the fiber onto the stent.

The connecting fiber (90) is wound, woven, tied, bonded, or attached to each segment and joins each segment with its neighboring segment. The connecting fibers (90) can be made of a polymeric material or a thin metal filament however the preferred embodiment for an SFA stent that is exposed to significant movement of the vessel is to form the connecting fiber (90) from a biodegradable material. The stent can be delivered to the vessel on a balloon delivery catheter. Once it reaches the site of the lesion, the stent is enlarged in diameter. The connecting fiber (90) holds the segments (45) in line with each other and prevents their embolization. After a few days, the stent segments (45) have been adequately healed into the vessel wall and the need for the connecting fibers (90) does not exist. Degradation or dissolution of the connecting fibers (90) allows each of the stent segments (45) to move freely with respect to each other. This will result in fewer strut fractures and less stresses being placed on the vessel wall and a better healing result for the vessel wall.

The struts (35) of the embodiment shown in FIGS. 1A, 1B, 3A, and 3B are nonparallel struts (120) in the non-deployed state owing to the tapered shape of each segment which extends from a larger outer diameter (125) to a smaller outer diameter (130). The inner surface (122) of one segment is overlapped by the outer surface (123) of its neighboring segment in the overlap region (115).

As shown in FIGS. 3A this stent (50) embodiment is also formed of tapered segments (92) that are overlapping each other thereby making the shape of each segment into a gradual conical surface during delivery. Although only two tapered segments (92) are shown in FIGS. 3A and 3B, it is understood that many such segments could extend in an axial direction (165) and are joined to one another via spacing members (55).

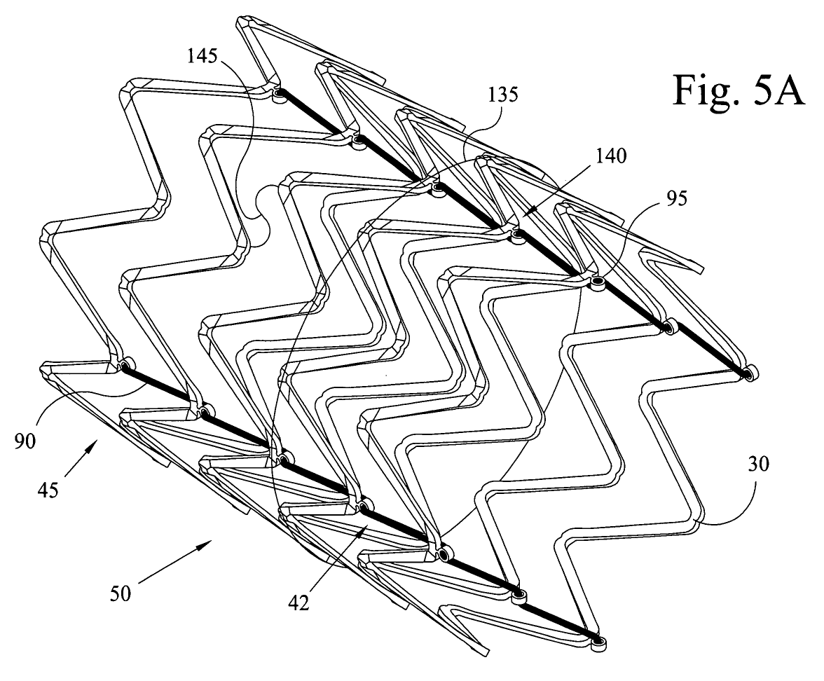

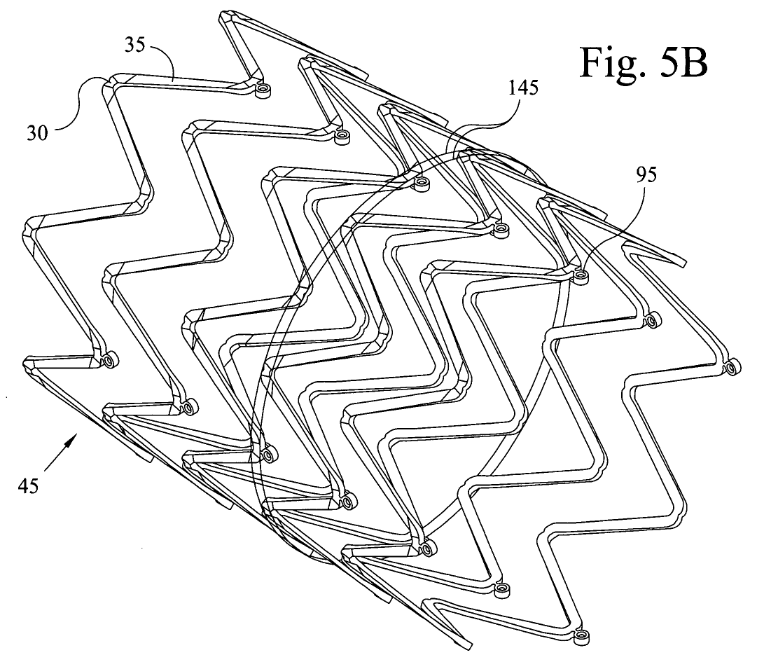

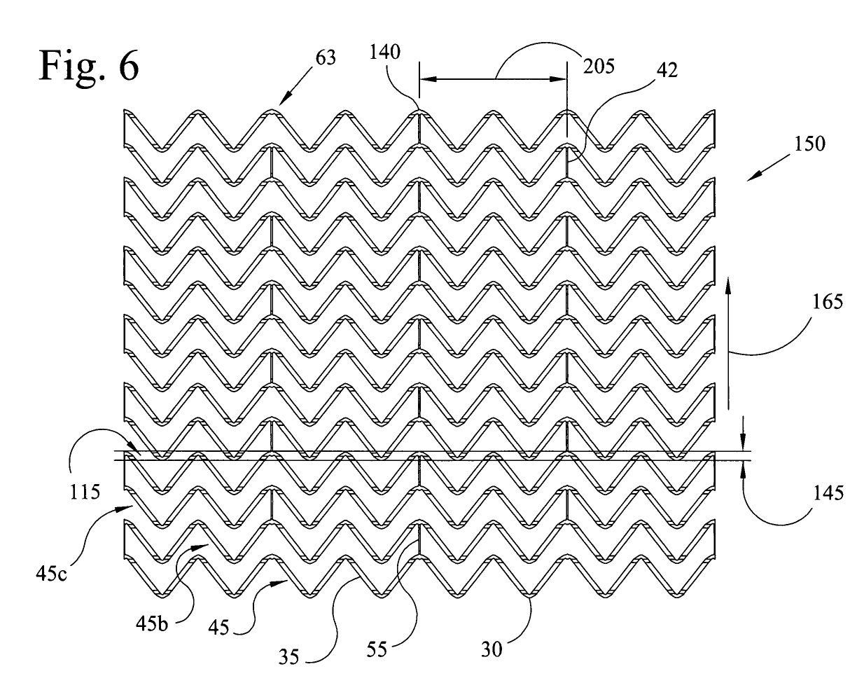

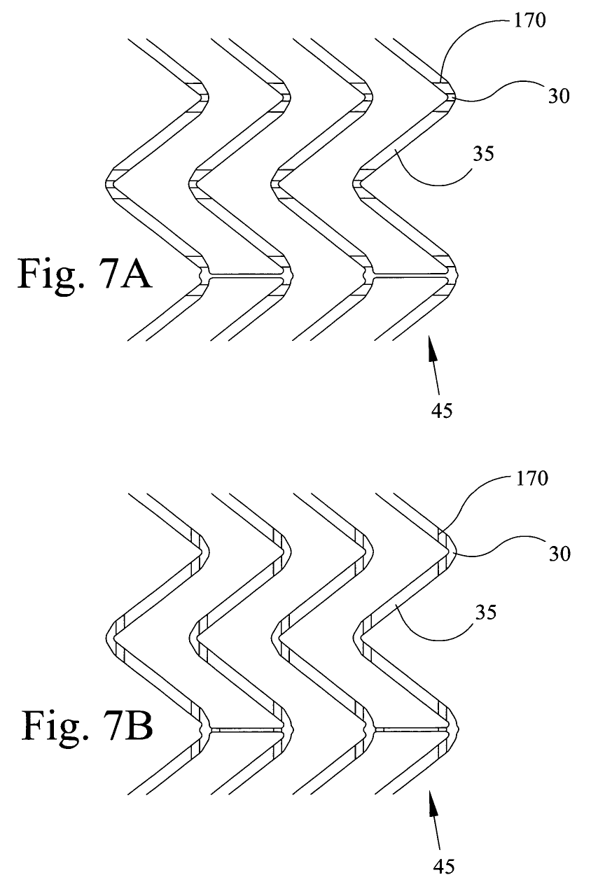

This overlapping provides two benefits to the stent (50). Overlapping allows the stent (50) to be more flexible in its nondeployed state because each segment can move relative to its neighboring segment (45) without the end of one segment (45) impinging into the end of another segment (45). Also, the overlapping allows the stent (50) to enlarge to a greater diameter and provide for better scaffolding because the peak (140) of one segment extends into the space identified by the hinge perimeter (135) of a neighboring segment (45) in a deployed state. This close positioning or nesting (145) of one segment relative to its neighbor is shown in one embodiment having connecting fibers (90) in FIGS. 5A and 5B and for the embodiment having spacing members (55) in FIG. 6. Other conformations for the connecting fibers (90) can also be adapted to the stent (50) of the current invention. One embodiment for the struts (35) is in the form of a modified ziz-zag pattern as shown in the deployed conformation in FIGS. 5A and 5B.

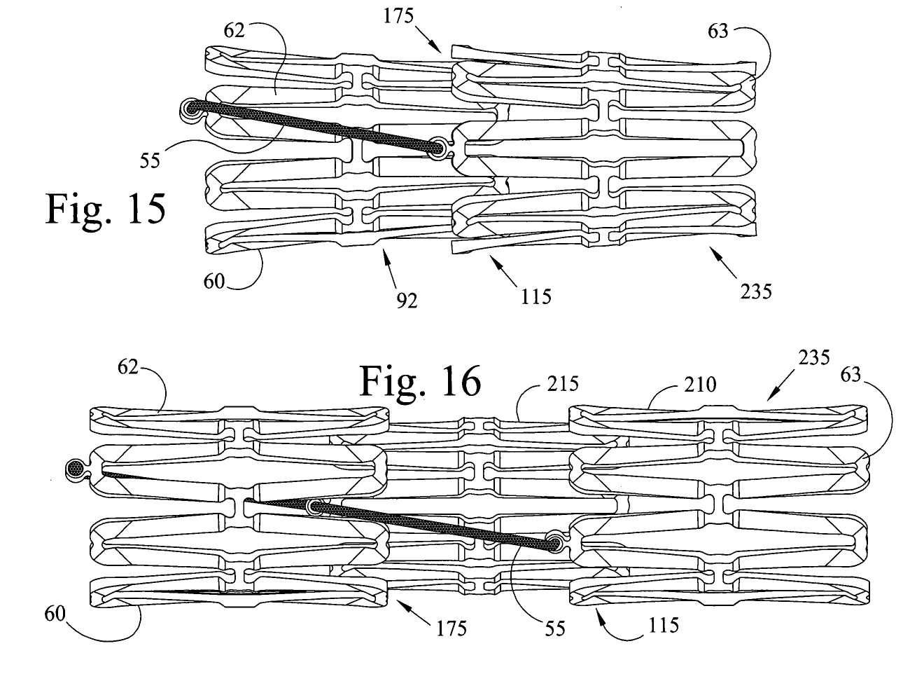

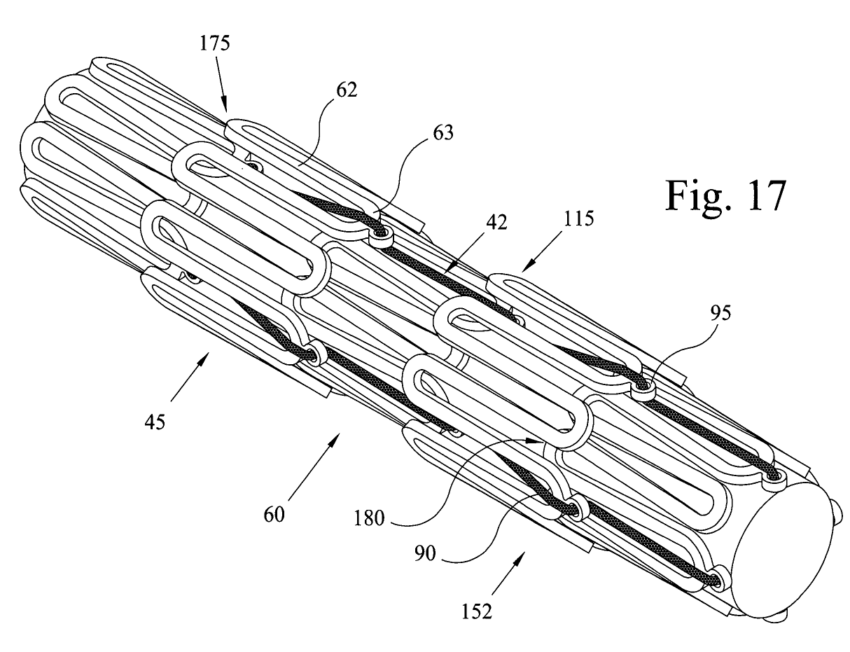

The conformation of the joining elements (42) that are either connecting fibers (90) or spacing members (55) of the embodiments shown in FIGS. 1A, 1B, 3A, and 3B attach the peak of one segment to the peak of its neighboring segment and is intended to not cause significant length change during deployment. It is understood that other geometries can be used to connect one segment to another that could result in length change during deployment. Also, the geometry shown in FIG. 6 is a modified zig-zag geometry (150) due to the presence of the hinge and strut design that was illustrated in FIG. 2 and the overlap region (115). Other geometries for the hinges (30) and struts (35) also are used including closed cell design (235), open cell design (see FIGS. 15 and 17), and combinations.

Connecting fibers (90) having generally a small cross-sectional area used to ensure that the segments (45) remain aligned and spaced evenly as they attach one segment with a neighboring segment. The cross-sectional dimension for these connecting fibers (90) can be approximately 0.0025 by 0.0025 inches and can range from 0.0015 to 0.005 inches and can be made of filaments (105) that can be as small as one tenth of the diameter of the fiber. The location of the connecting fibers (90) for one embodiment can be seen in the deployed state in FIG. 5A. Other connecting fiber orientations can be used in the stent of the present invention. FIG. 5B shows the expanded state with the connecting fibers dissolved or degraded and therefore not present.

Other conformations for the spacing members (55) can also be adapted to the stent (50) of the current invention. One embodiment for the struts (35) is in the form of a modified zig-zag pattern as shown in the deployed conformation in FIG. 6. The segments (45) are joined together in the axial direction (165) via spacing members (55). The struts (35) are joined via transition regions (see FIG. 2) and hinges (30) to other struts (35). The transition region (see FIG. 2) forms a smooth transition from the strut (35) which has a small radial dimension and large width to the hinge (30) which has a large radial dimension and small width. Nesting (145) allows the peak of one segment to reside closer in an axial direction (165) within the space occupied by a neighboring stent segment (45).

Spacing members (55) having generally a small cross-sectional area ensure that the segments (45) remain aligned and spaced evenly and attach one segment (45) with a neighboring segment (45b) on its right side 180 degrees across from each other. Other spacing members (55) attach that segment to a neighboring segment (45c) forming a 90 degree phase angle (205). The spacing members (55) can be straight as shown in FIG. 3A or they can be curved as shown in FIG. 3B to allow for extension deformation as the stent (50) is exposed to a bending deformation. The cross-sectional dimension for these spacing members (55) can be approximately 0.0025 by 0.0025 inches and can range from 0.0010 to 0.005 inches. The location of the spacing members (55) can be seen also in the deployed state in FIG. 6 with the 90 degree phase angle (205) from one spacing member pair to the next. Other phase angles and spacer member orientations can be used in the stent of the present invention.

For those applications where the movement of the vessel causes significant stent deformation such as bending, twisting, or stretching the use of very flexible spacing elements with smaller dimensions would allow each segment to move very independently from its neighboring segment. If such a spacing element should break or fracture, the amount of inflammation associated with the flexible and small cross-sectional dimension spacing element would be less than that associated with a more rigid connector found in the self-expanding stents currently being used in the SFA, popliteal artery, other arteries of the leg, and veins.

The transition region (40) provides a gradual dimensional change from the strut (35) to the hinge (30). The strut-transition line (170) is shown in FIG. 1A to allow for ease of machining the outer surface (123) of the stent (50). The outer surface (123) is intended to be machined with the struts (35) in an intermediate position as shown in FIGS. 7A and 7B which is larger than the nondeployed diameter as shown in FIGS. 1A, 1B, 3A, and 3B yet smaller than the fully deployed diameter as indicated by FIGS. 5A or 6. The stent (50) outer surface (123) is machined without overlap of the two segments (45) as shown in FIGS. 7A and 7B. The circumferentially machined strut transition line (170) shown in FIG. 7B will produce the strut-transition line (170) shown in FIG. 1A. An alternate transition line can be machined with an axial alignment as shown in FIG. 7A.

As shown in FIG. 3A the stent (50) has a stepped outer surface (175) and a stepped inner surface (180) in its nondeployed state due to the overlapping of one segment over a portion of its neighboring segment. Since the stent (50) is intended to be delivered via a balloon catheter, a balloon will be positioned under the inner surface (122) of the stent (50). Allowing the balloon material to extend into this stepped inner surface (180) will allow the stent (50) to be held more securely to the balloon in a deliverable or nondeployed state. This will be of benefit to ensure that the stent does not become dislodged during placement within the stenotic lesion in the blood vessel and does not dislodge if the stent and catheter is withdrawn back into the guide catheter that is used to deliver the stent to the site of the lesion.

As shown in FIGS. 1A, 1B, 3A, and 3B the outer surface (123) additionally has protuberances (185) associated with the increased height of the hinges (30) in comparison to the strut (35). These protuberances (185) will help to seat into the vessel wall and assist with anchoring of the stent (50). Additionally, the insertion of a small protuberance into the vessel wall during implant can act as sites for accessing healthy tissue located beneath the surface deposits found on a vessel surface to be brought to the lumen and assist with healing of the vessel lesion. During delivery of the stent (50) to the lesion, these protuberances (185) may catch on a previously placed stent or on an edge of a delivery catheter. The hinge edges can be tapered to improve the leading edge and reduce snagging

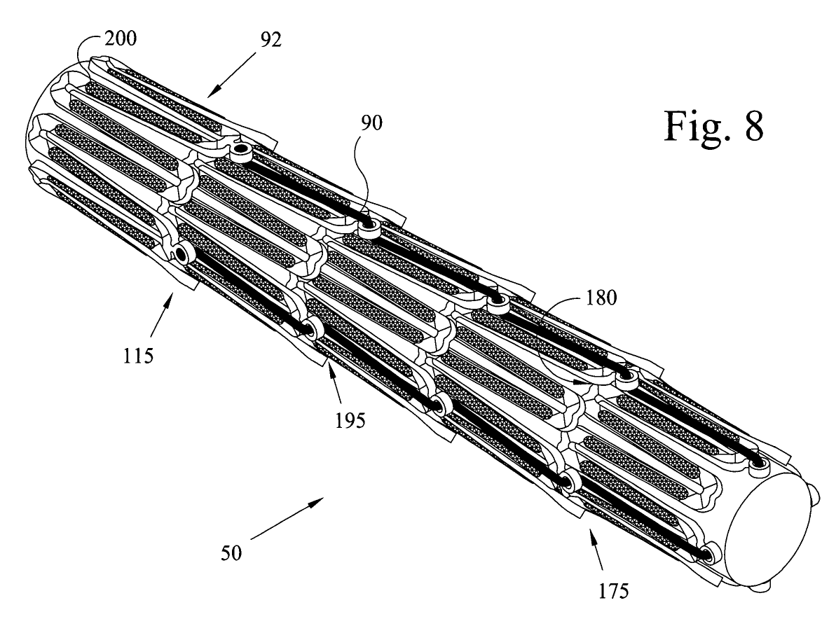

FIG. 8 shows a perspective view of the end of the stent (50) with the tapered segments (92), the overlap region (115), and the connecting fibers (90) that join a segment with neighboring tapered segments (92). The stepped outer surface (175) and stepped inner surface (180) can be seen in this view. The overlap of one segment over the next creates a radial gap (195) between the hinge of one segment and the strut of its neighboring segment. This radial gap (195) help provide flexibility to the stent (50) as it is exposed to a bending deformation by allowing space for movement without impacting one segment (45) against its neighboring segment (45).

FIG. 8 also shows the presence of a drug or drug/polymer coating (200) located on the outside of a strut of this balloon-expandable embodiment. The drug/polymer coating (200) can be a restenotic drug such as paclitaxel or sirolimus or a biocompatible polymer coating that resists thrombosis and inflammation. Due to the small radial dimension for the strut, the drug and coating can be applied to the strut (35) without affecting the profile of the stent (50). The drug can also be applied to other surfaces of the present stent (50). The drug or drug/polymer combination can be applied to the struts (35) of any of the embodiments of the present invention. Those embodiments that have the hinge and strut structure as shown in FIG. 8 can be made to be balloon-expandable and non-crushable. Delivering such a stent (50) on a balloon catheter rather than within an external sheath obviates the scraping of the drug and polymer associated with removal of the sheath from a self-expanding stent system.

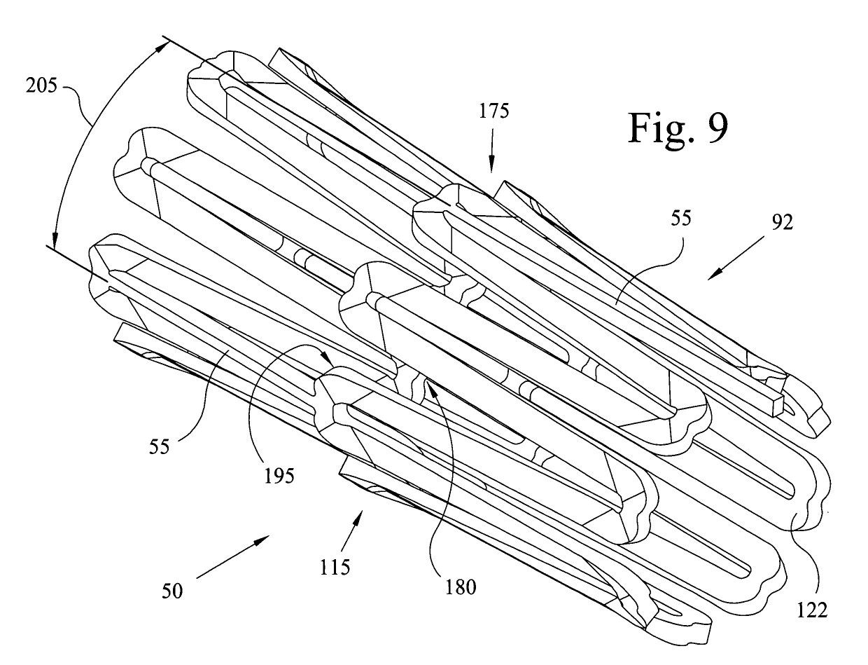

FIG. 9 shows a perspective view of the stent (50) with the tapered segments (92), the overlap region (115), and the 90 degree phase angle (205) between the spacing members (55) that join a segment with neighboring tapered segments (92) on one end versus the other end of the segment. The stepped outer surface (175) and stepped inner surface (180) can be seen in this view. The overlap of one segment over the next creates a radial gap (195) between the hinge of one segment and the strut of its neighboring segment. This radial gap (195) helps provide flexibility to the stent (50) as it is exposed to a bending deformation by allowing space for movement without impacting one segment (45) against its neighboring segment (45).

FIG. 5A and 6 shows the stent (50) with joining elements (42) that are either connecting fibers (90) or spacing members (55), respectively, in its final deployed conformation with a portion of one segment extending close or nesting (145) within the space of an adjacent segment. In this deployed conformation the stent (50) is very flexible because each segment can move well without significantly affecting the neighboring segment. This freedom of movement between each segment will also provide a stent (50) with reduced strut fracture failure due to vessel movements. As shown in this figure the stent (50) will not undergo significant length change from its nondeployed to its deployed state. The lack of foreshortening is accomplished by connecting the peaks (140) of one stent segment (45) with similarly directed peaks (140) of a neighboring segment (45).

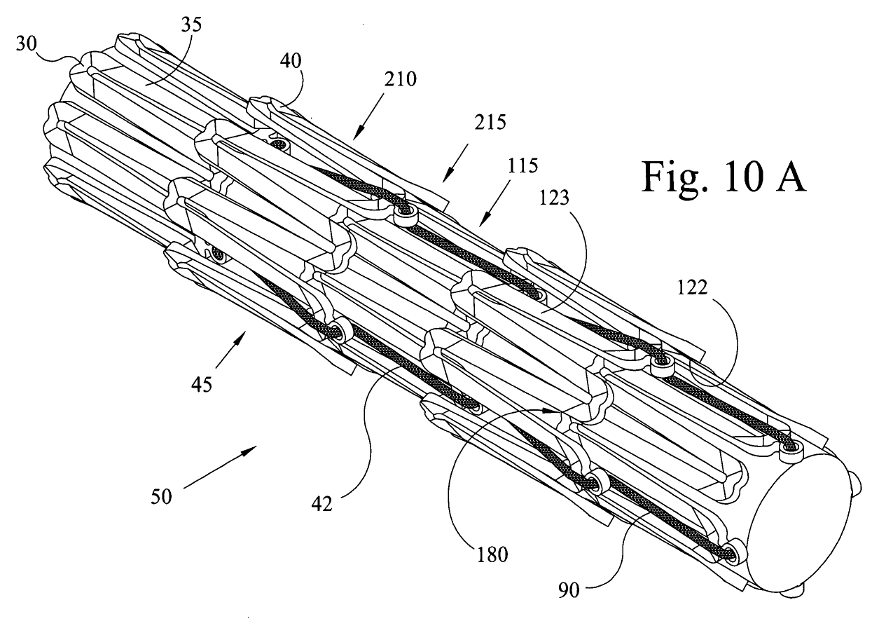

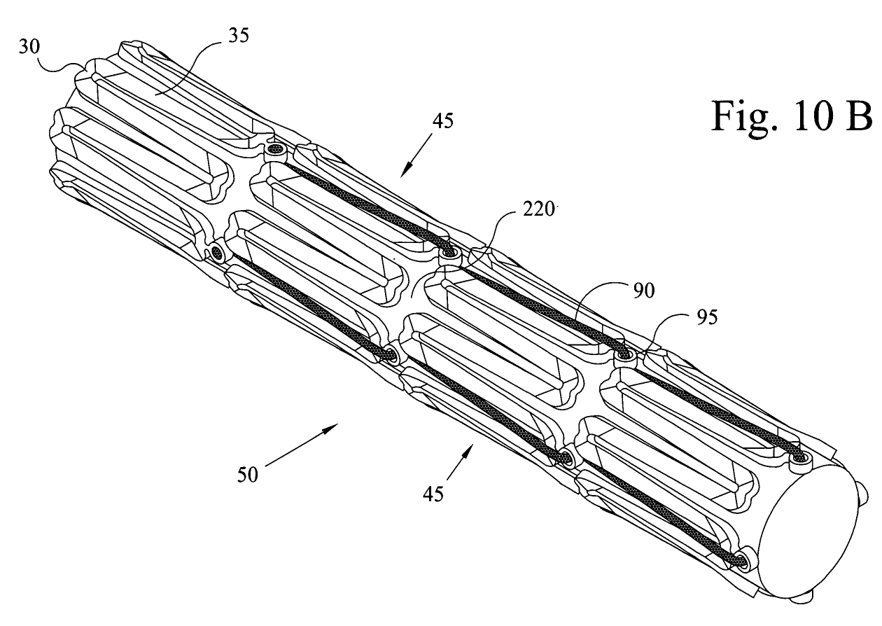

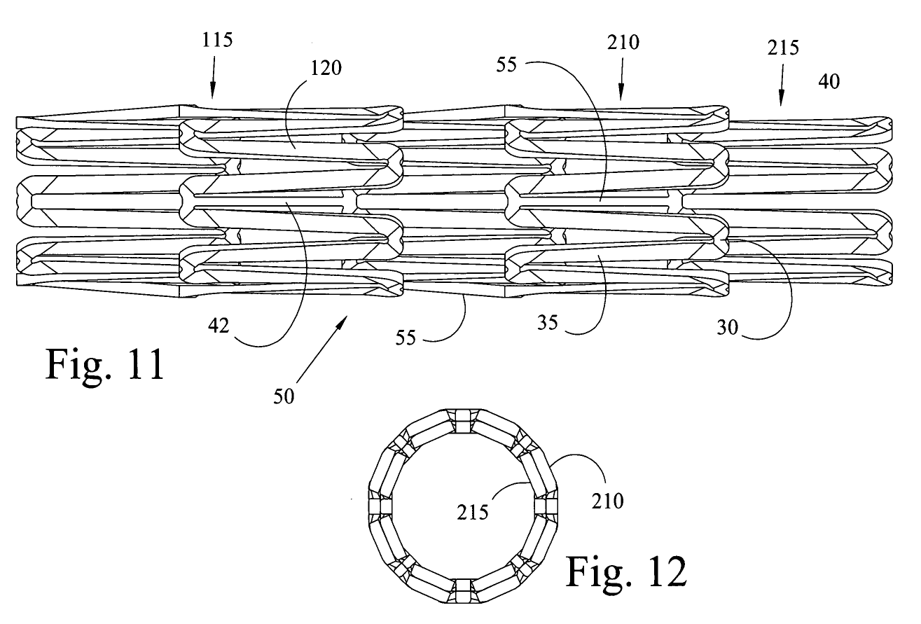

An alternate embodiment of the present invention where the joining elements (42) are connecting fibers (90) as shown in FIG. 10A and are spacing members (55) as shown in FIG. 11. Each segment of the stent (50) has the hinge (30), strut (35), and transition region (40) constructions that were described earlier. FIGS. 10A and 11 show a large diameter outer segment (210) joined to a smaller diameter inner segment (215) via a connecting fiber (90) or spacing member (55) in the non-deployed state. The struts (35) on the outer segments (210) can be generally nonparallel to each other as they have been forced into a position over the inner segment (215) during delivery, and the struts (35) of the inner segments (215) can be generally more parallel to each other as shown in this embodiment; alternately the parallel and nonparallel struts (120) can be reversed. Connecting fibers (90) shown in FIG. 10A attach an inner segment (215) to an outer segment (210) on one of its ends, and connecting fibers (90) attach that inner segment (215) to another outer segment (210) on the other of its ends. The connecting fibers (90) can be biodegradable, polymeric nondegradable, or metallic. The deployed state of this stent (50) is similar to that shown in FIG. 5A.

As shown in FIG. 10A the neighboring segments (45) can be of two different diameters such that the larger diameter outer segment (210) overlaps with smaller diameter inner segments (215) on each side of it. Every other segment is either of a larger diameter or a smaller diameter. The outer surface (123) of the smaller diameter segment is in close approximation to the inner surface (122) of the larger diameter segment in the overlap region (115). The overlap regions (115) provide this stent (50) with improved flexibility in the nondeployed state and allow the stent (50) to have improved scaffolding in the deployed state. The embodiment shown in FIG. 10A has a similar capability to secure to an underlying balloon during delivery due to the stepped inner surface (180) and possesses other advantages and characteristics that have been described for the embodiment shown in FIG. 1A and 1B.

In FIG. 11 the spacing members (55) attach an inner segment (215) to an outer neighboring segment (210). Spacing members (55) attach that inner segment (215) to another outer segment (210) 180 degrees across from each other. The spacing members (55) on one end of an inner segment (215) form a 90 degree phase angle (205) (see FIG. 6) with the spacing members (55) on its other end. The deployed state of this stent (50) is similar to that shown in FIG. 6. FIG. 12 shows an end view of the present embodiment having an inner segment (215) and an outer segment (210).

FIG. 10B shows another embodiment of a stent (50) structure similar to FIG. 10A except that it has axial space (220) or axial gaps between each neighboring segment (45). The axial space (220) shown in FIG. 10B provides flexibility to the stent (50) during delivery as the stent (50) is exposed to a bending deformation. Connecting fibers (90) again are used to join adjacent segments (45) together. During delivery and deployment of this stent (50) via a balloon catheter, the connecting members do not require a compressive strength and therefore can be flexible.

The wall structure for the stent (50) of the present invention is not limited to that described in FIGS. 1A-12. Other embodiments having axial overlap regions (115) and alternate geometries such as closed cell geometries, other open cell geometries, or combinations for segments (45) formed from hinges (30) and struts (35) and joined via spacing members (55) are also anticipated.

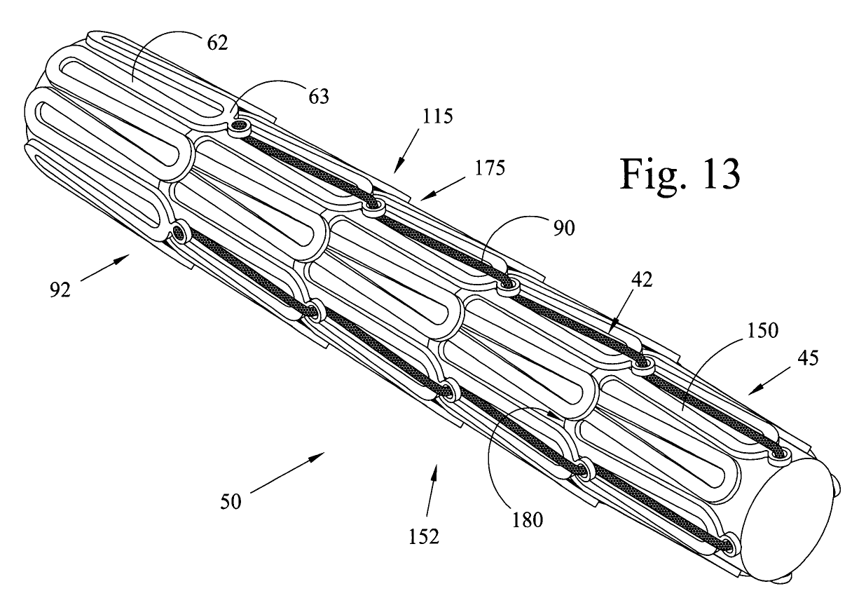

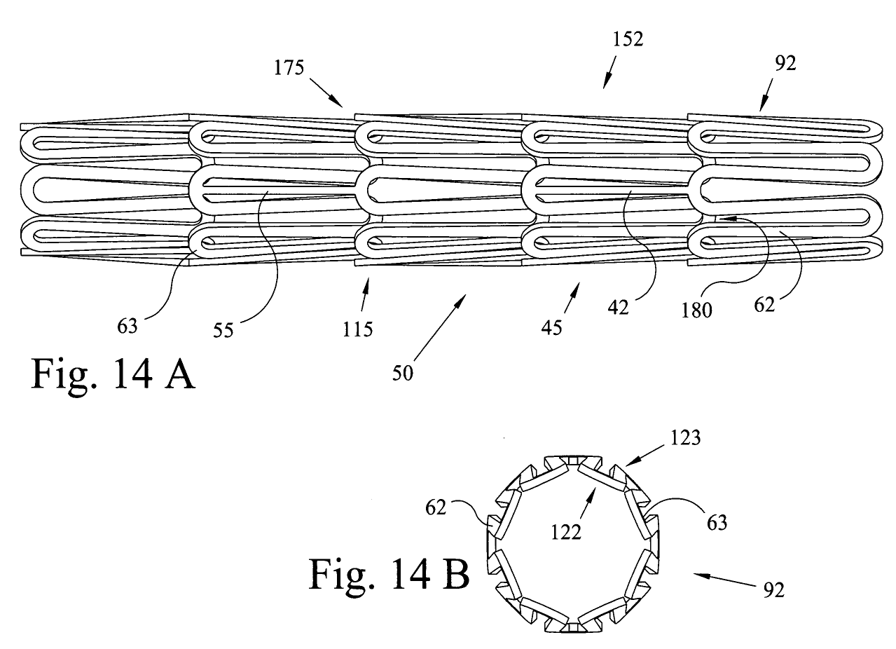

FIG. 13 shows another embodiment for the present invention applying the axial overlap of neighboring segments (45) to a stent (50) having a more standard wall structure; i.e., one having elongated elements (62) and junctional regions (63) rather than struts with thin radial dimension and large strut width and hinges with large radial dimension and a small hinge width. As shown in FIGS. 13 and 14A, the joining elements (42) can be connecting fibers (90) or spacing members (55) used to join neighboring segments (45) to form a single stent (50). The overlapping provides the advantage of a greater scaffolding of the vessel wall in its deployed state. The configuration in the deployed state can be more closely nested in a way that resembles the nesting (145) shown in FIGS. 5A or 6. The overlapping also provides more flexibility to the stent (50) during delivery by preventing the ends of each segment from impinging upon the end of its neighboring segment when it is placed into a bent conformation.

Embodiments for either a balloon-expandable or self-expanding stent without the hinge and strut structure described earlier in FIG. 2 can have the geometry of a modified zig-zag structure (150) like the embodiments shown in FIGS. 13 having the connecting fibers (90), or in FIGS. 14A and 14B for the embodiment having spacing members (55). Junctional regions (63) provide the junction between one elongated element of a stent and another elongated element. Each segment is tapered and lies below its neighboring segment on one side and above its neighboring segment on the other. An overlap region (115) is present and creates a stepped outer surface (175) and a stepped inner surface (180). The stepped inner surface (180) can assist in holding a balloon-expandable stent more securely against its underlying balloon in the non-deployed state. Connecting fibers (90) join each segment with its neighboring segment. Alternately the geometry can be even more similar to the standard zig-zag structures found in many of the stents currently used in the clinic. An example of standard zig-zag structure (152) being applied to two stent embodiments of the present invention having overlapped segments (45) and either connecting fibers (90) or spacing members (55) is shown in FIGS. 13 and 14A. The geometry for the wall for each segment can also be a closed cell design (235) (see FIGS. 15 and 17), or it can be a composite of an open cell and a closed cell design. FIG. 14B shows an end view of the embodiment of FIG. 14A showing a tapered segment (92).

The geometry for the wall for each segment can also be a closed cell design (235), an example of which is shown in FIG. 15 with tapered segments (92). The wall structure can also be a composite of an open cell and a closed cell design (235). FIG. 16 shows a geometry for a closed cell design (235) with a zig-zag structure having spacing members and a stepped outer surface (175). The wall structure (60) has large diameter outer segments (210) and small diameter inner segments (215) comprised of elongated elements (62) joined at junctional regions (63).

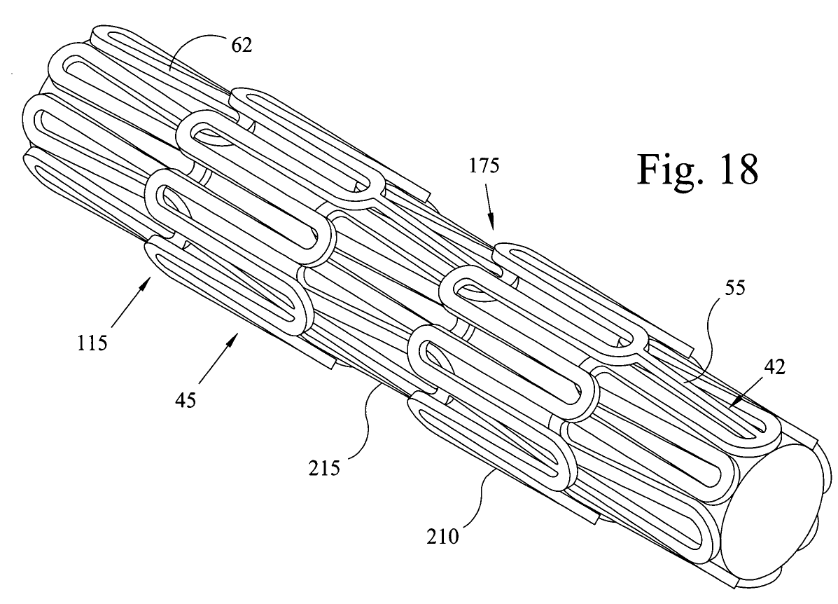

Additional embodiments of a standard stent (50) structure having joining elements (42) such as connecting fibers (90) as shown in FIG. 17 and having spacing members as shown in FIG. 18. FIGS. 17 and 18 show open cell designs for the wall structure. Each of the segments (45) shown in FIGS. 17 and 18 are generally cylindrical in shape. Each segment (45) is joined to its neighboring segment (45) by a connecting fiber (90) or a spacing member (55), respectively. It is understood that the wall structure for the present invention can be an open cell such as a zig-zag, a closed structure, or a combination. Many forms of zig-zag patterns are also anticipated for the wall structure.

Either the cylindrically shaped segments (45) or the tapered segments (92) can be formed of a wall geometry that is an open cell design, a closed cell design, or a combination of the two. The stent of this embodiment without the specific hinge and strut structure described in FIG. 2 can be either self-expanding or balloon-expandable. If it is self-expanding, the material for the stent elongated element and junctional region could be Nitinol, Elgiloy, or other elastic metal or alloy. For a balloon-expandable stent the material could be stainless steel, titanium, platinum, or other metal that will plastically deform upon expansion by the balloon delivery catheter over which it is mounted. Similar advantages exist for the securement for either stent onto a balloon of a delivery catheter due to the stepped inner surface created by the overlap region. Also biodegradable materials such as polyethylene glycol, polyglycolic acid, polylactic acid, copolymers of polycarbonate, and other biodegradable polymers and biodegradable metals including magnesium can be used to form the elongated elements and junctional regions of the stent. Similar materials can also be used to form a self-expanding stent.

In an expanded state, the overlap region is no longer overlapped but the overlap which is present during delivery allows the stent to have a greater scaffolding in a deployed state. This greater scaffolding is provided by creating a closer nesting between neighboring segments in a deployed state as described earlier. This overlapping can be applied to almost all stent structures to enhance the amount of scaffolding provided to the vessel wall.

Although the stent embodiments described herein have advantages that are associated with a balloon-expandable stent, the invention also includes the use of overlapping segments (45), connecting fibers (90), and spacing members (55) in self-expanding stents. The dimensions for the hinges (30) and struts (35) would be adjusted to provide for hinges (30) remaining elastic during an expansion deformation. The hinge length (65) for a self-expanding stent would be larger than for a balloon-expandable stent. The use of overlap regions (115) in order to improve flexibility during delivery and scaffolding after the stent is deployed has application to both balloon-expandable and self-expanding stents. The use of a biodegradable fiber or a flexible fiber to provide independent movement of each segment with less fatigue fracture problems also has application to both balloon-expandable and self-expanding stents. It is understood that the concepts described in this application are not limited to the embodiments presented but can be applied to other stent designs also.

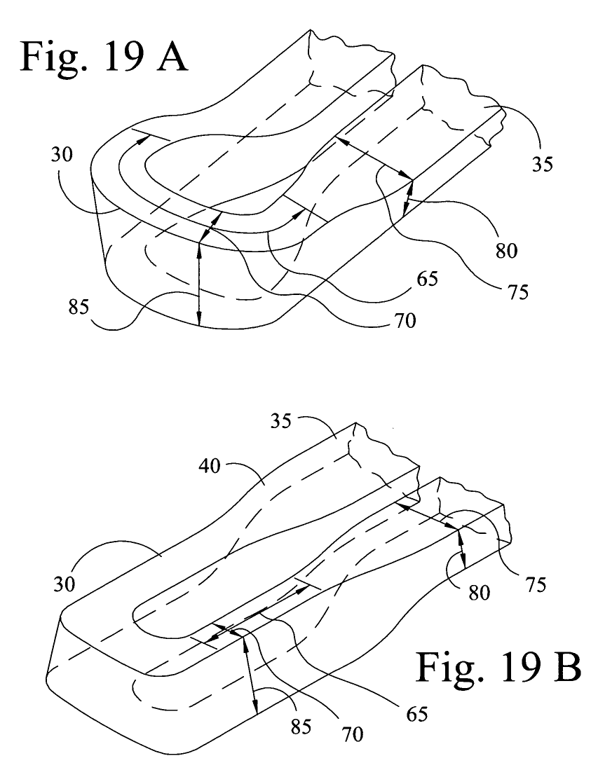

FIG. 19A shows the hinge (30) and strut (35) which form the wall structure (60) of a stent (50) that is self-expanding and is able to have a tapered overlap structure as shown if FIG. 3A or a parallel overlap structure as shown in FIG. 11. To provide the stent (50) with self-expanding characteristics the hinge length (65) is enlarged so that the expansion deformation is not focused. The hinge width (70) is smaller than the strut width (75) to ensure that the expansion deformation occurs only in the hinge region. The hinge width (70) and radial dimension are larger than the strut radial dimension (80) to provide an expansion force that is tailored to the desired level. The strut has a wide width and thin radial dimension as described earlier.

FIG. 19B shows another embodiment for the hinge (30) and strut (35) wall structure (60) for a stent that is self-expanding. The strut (35) has a strut width (75) and strut radial dimension (80) that is similar to that described in FIG. 19A. The wall structure (60) can have two hinges (30) each of which has a hinge width (70) that is narrower than the strut width (75) and a hinge radial dimension (85) that is larger than the strut radial dimension (80). The hinge length (65) is longer than the hinge length for a balloon-expandable wall structure (60) such as shown in FIG. 2. The longer hinge length (65) as shown in this embodiment does not focus the deformation associated with the expansion of the stent. By shortening the hinge length (65) this wall structure (60) having two hinges associated with junctional region (63) can also be a wall structure for a balloon-expandable stent.

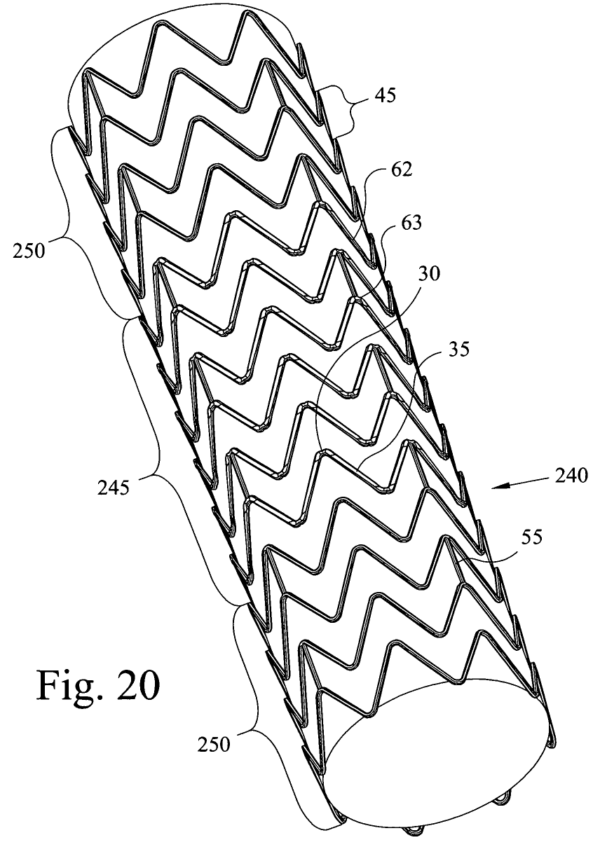

FIG. 20 shows an embodiment of a composite stent (240) of the present invention in its deployed configuration. The composite stent (240) has a centrally located balloon-expandable region (245) and two self-expanding regions (250), one located at each end of the stent. The balloon-expandable region (245) is comprised of hinges (30) and struts (35) that are the same as those described in FIG. 2. Each segment of the balloon-expandable region (245) can be connected together via joining elements (42) that are either connecting fibers (90) or via spacing members (55) as shown for example in FIGS. 10B, 13, or 18. The segments (45) can be overlapped (not shown) in its non deployed configuration as described earlier or not overlapped and can also have an open cell or closed cell structure as shown earlier. At each end of the composite stent (240) is located a self-expanding portion which can be constructed via a standard zig-zag construction that can be open cell as shown or closed cell. The standard zig-zag construction can be any self-expanding stent wall structure (60) currently being used or anticipated for stents. The self-expanding portions (250) can be joined contiguously to the balloon-expandable portion (245) via spacing members (55). Alternately, connecting fibers (90) can join individual segments (45) of the self-expanding portions (250) together and can join the self-expanding portions (250) to the balloon-expandable portion (245).

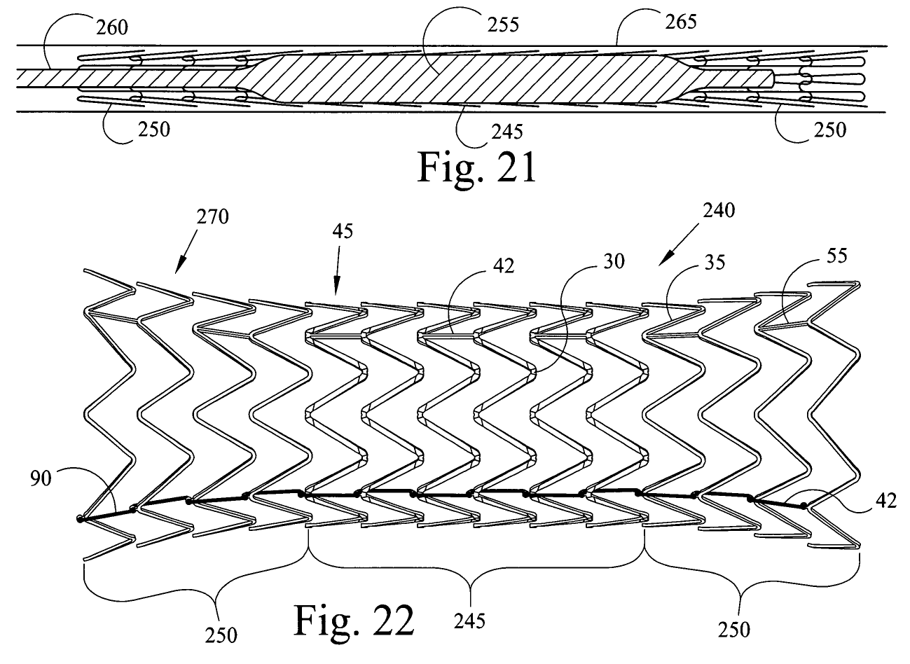

The composite stent (240) is delivered to the vessel or tubular member of the body with the balloon-expandable portion (245) loaded onto a dilatation balloon (255) of a dilatation catheter (260) as shown in FIG. 21. The self-expanding portions (250) are held downward in a nondeployed configuration by an external sheath (270). Delivery of the stent requires that the external sheath (270) is withdrawn releasing the self-expanding portions (250). These self-expanding portions (250) expand outward with a very small outward force but expand to a very large diameter to make contact with the wall of the vein or other tubular member to ensure that the device does not embolize. The dilatation balloon (255) is then expanded to force the balloon-expandable portion (245) of the composite stent (240) out to its nominal diameter. The balloon-expandable portion (245) has its struts designed to allow some ovality to occur to generally match the external forces being placed upon it without crushing. In the case of an iliac vein being subjected to compression syndrome of the iliac artery, the balloon-expandable portion (245) is designed to have similar restraining force to match that being imposed by the neighboring iliac artery.

As the composite stent (240) is released into the vessel or tubular member of the body it expands outward to form a shape that is similar to that shown in FIG. 22. The self-expanding portions (250) extend outward to a larger extent forming a funnel shape (270) to ensure contact with the varying diameters of a venous wall. The central balloon-expandable portion (245) maintains a perimeter that is set by the properties of the hinges (30). The area maintained for blood flow would be set to ensure that thrombosis due to reduced flow area did not occur. The segments (45) can be joined together via joining elements (42) which can be either spacing members (55) or connecting fibers (90) or a combination of both applied to any portion of the stent.

It is understood that the wall structures described in the embodiments of this invention can have two or more hinges associated with a junctional region and can have two or more struts entering into a junctional region. The length of the hinges can be adjusted to make the wall structure either balloon-expandable or self-expanding. The invention is not intended to be limited to the embodiments discussed herein.

We claim:

1. A stent that is delivered to a tubular vessel of the body in a small diameter state and enlarged to a larger diameter state within a tubular vessel of the body, the stent comprising;
A. segments that are joined by joining elements,
B. said segments having elongated elements and junctional regions,
C. said stent having an overlap region shared between at least two neighboring segments.

2. The stent of claim 1 wherein said joining elements are connecting fibers.

3. The stent of claim 2 wherein said connecting fibers are biodegradable.

4. The stent of claim 1 wherein said joining elements are spacing elements that are more flexible than said elongated elements.

5. The stent of claim 1 wherein said stent is balloon-expandable and is thereby delivered to the vessel on a balloon that is located on a catheter.

6. The stent of claim 1 wherein said stent is self-expanding and is adapted to be released from its smaller diameter state by removing an external sheath.

7. The stent of claim 1 wherein said joining elements do not have significant compressive strength such that they are unable to significantly resist axial length reduction between neighboring segments.

8. The stent of claim 1 wherein said elongated elements are struts and said junctional regions are hinges, said hinges having a hinge width smaller than the width of said struts and said struts having a radial dimension that is smaller than the radial dimension of said hinges, the hinge length being less than twice the hinge width to provide the stent with characteristics to be balloon-expandable and non-crushable.

9. The stent of claim 1 wherein said elongated elements are struts and said junctional members comprise one or more hinges, said hinges having a hinge width smaller than the width of said struts and said struts having a radial dimension that is smaller than the radial dimension of said hinges, said hinge length being greater than twice the hinge width to provide the stent with characteristics to be self-expanding.

10. The stent of claim 8 wherein a drug is placed on the surface of said stent to reduce stent restenosis.

11. The stent of claim 1 wherein the external surface of said stent is a stepped surface.

12. The stent of claim 1 wherein said segments are tapered; said tapered segments extending over a portion of a neighboring segment and extend under a portion of another neighboring segment.

13. The stent of claim 1 wherein said segments are of two diameters, a smaller diameter and a larger diameter, said smaller diameter segment extends under a portion of each neighboring larger diameter segments forming an overlap region between segments.

14. The stent of claim 1 wherein the deployed state has segments with peaks that lie within the perimeter formed by peaks of its neighboring segment to provide nesting for improved scaffolding of the vessel.

15. The stent of claim 9 wherein said junctional region comprises two or more hinges.

16. The stent of claim 1 having one or more portions of said stent that is self-expanding with self-expanding segments and having one or more other portions of said stent that is balloon-expandable with balloon-expandable segments.

17. The stent of claim 16 wherein said balloon-expandable portion has elongated elements that are struts and has junctional regions that are hinges, said hinges having a hinge radial dimension that is greater than a radial dimension of said strut and said hinge having a hinge width that is less than a width of said strut.

18. The stent of claim 16 wherein said self-expanding portion has an outward force in its large diameter state that is low enough to prevent migrate through a wall of a tubular body vessel but has a large diameter state that will contact the wall of the tubular vessel.

19. The method of delivery for a stent to a tubular vessel of the body in a smaller diameter state and enlarged to a larger diameter within a tubular vessel of the body, said method comprising;
A. placing a stent having segments that are joined by joining elements, and having at least one overlap region between adjacent segments, upon a balloon dilatation catheter and having an external sheath placed around said stent,
B. removing the external sheath from at least a portion of said stent to allow at least a portion of said stent to enlarge to a larger diameter,
C. inflating the balloon on the balloon dilatation catheter to dilate at least a portion of said stent,
D. removing the external sheath and balloon dilatation catheter from the tubular vessel.
